(19) United States
(12) Patent Application Publication
(10) Pub. No.: US 2016/0074422 A1
(43) Pub. Date: Mar. 17, 2016

(54) MUCUS STRENGTHENING FORMULATIONS TO ALTER MUCUS BARRIER PROPERTIES

(71) Applicant: NORTHEASTERN UNIVERSITY, Boston, MA (US)

(72) Inventors: Rebecca Lyn Carrier, Boston, MA (US); Hasan Mahmut Yildiz, Boston, MA (US)

(21) Appl. No.: 14/787,550

(22) PCT Filed: May 8, 2014
(86) PCT No.: PCT/US14/37357

§ 371 Date: Oct. 28, 2015

Related U.S. Application Data
(60) Provisional application No. 61/820,946, filed on May 8, 2013.

(51) Int. Cl. A61K 31/685; A61K 47/44; A61K 45/06; A61K 47/12; A61K 47/14; A61K 31/575; A61K 47/02

(52) U.S. Cl. CPC A61K 31/685; A61K 31/575; A61K 47/44; A61K 47/02; A61K 47/12; A61K 47/14; A61K 45/06

(57) ABSTRACT

The present technology relates to compositions and methods that strengthen mucus barriers, e.g., reduce the permeability of the mucus barriers. In some embodiments, the compositions include a model bile and a lipid mixture, wherein the model bile includes at least one salt and a lecithin. In some embodiments, the composition is useful to treat necrotizing enterocolitis.

### CROSS-REFERENCE TO RELATED APPLICATIONS

This application claims priority to U.S. Application No. 61/820,946 filed May 8, 2013, the content of which is hereby incorporated by reference in its entirety.

### GOVERNMENT FUNDING

This invention was made with U.S. Government support under grant 1R21EB015750 awarded by the National Institutes of Health. The U.S. Government has certain rights in the invention.

### BACKGROUND

Mucus is a complex gel network that forms a barrier between epithelial surfaces and the external environment. Mucus includes a highly branched protein network, the major structural component of which is mucin glycoproteins. Mucus also contains ˜2% (by mass) lipids and 90-95% by mass salts, cells, electrolytes, other cellular debris and water. Mucus functions as a robust barrier to control the passage of harmful molecules and organisms such as bacteria and functions to facilitate efficient transport of nutrients across the epithelium.

### SUMMARY

In one aspect, the present technology relates to a composition including a model bile and a lipid mixture, wherein the model bile comprises at least one salt and a lecithin. In some embodiments, the at least one salt is selected from the group consisting of: bile salts, calcium-containing salts, sodium taurodeoxycholate (NaTDC), calcium chloride (CaCl₂), acetate, carbonate, chloride, glubionate, gluceptate, gluconate, lactate, lactobionate, phosphate salts, NaCl, KCl, NaOH, Triz-maleate, bile salts, e.g., sodium glycocholate (NaGC), sodium glycochenodeoxycholate (NaGCDC), sodium glycodeoxycholate (NaGDC), sodium taurocholate (NaTC), sodium chenodeoxycholate (NaCDC), sodium taurodeoxycholate (NaTDC), and porcine bile extract.

In some embodiments, the lecithin comprises one or more compounds selected from the group consisting of: phosphoric acid, choline, fatty acids, glycerol, glycolipids, lysolecithin, triglycerides, and phospholipids. In some embodiments, the lipid mixture comprises one or more of soybean oil, sodium oleate, and 1-oleoyl-rac-glycerol, and long chain triglycerides (e.g., monoolein and oleic acid), glycerol, glyceryl, olive oil, self-emulsifying drug delivery systems (SEDDS) (e.g., various formulations composed of vitamin E, Tween 80, labrasol, captex 355, ethyl alcohol in different proportions), self-emulsifying drug delivery systems (SNEDDS)(e.g., 30% w/w sesame oil, 30% w/w maisine 35-1, 30% w/w cremophor RH 40, 10% w/w ethanol), or a combination thereof.

In some embodiments, the composition also includes a pH lowering agent. In some embodiments, the composition also includes a maleate buffer.

In some embodiments, the model bile also includes cholesterol and lysophosphadylcoline.

In some embodiments, the composition also includes one or more biological agents, wherein the biological agent is selected from the group consisting of mucin, oligosaccharides, trefoil factor 3 (TFF3), resistin-like molecule-β (RELM-β), RegIIIγ, anti-microbial peptides (AMPs), lysozyme, lactoferrin, immunoglobulin A (IgA), immunoglobulin G (IgG), arachidonic (AA), docosahexaenoic acid (DHA), lactoferrin, and apo-lactoferrin.

In some embodiments, the ratio model bile to lipid mixture is about 4:1.

In another aspect, the present technology relates to methods for decreasing the permeability of a mucus barrier, the method comprising administering an effective amount of any of the above compositions to a subject.

In some embodiments, the mucus barrier is in the gastrointestinal tract, the respiratory airway, the cervicovaginal surface, or ocular surface. In some embodiments, the administration of the composition is parenteral, oral, inhalation, transdermal, intraocular, iontophoretic, or transmucosal. In some embodiments, the subject is human.

In another aspect, the present technology relates to methods for prevent or treating a defective mucosal barrier disease or disorders, the method comprising administering an effective amount of any of the above compositions to a subject in need thereof.

In some embodiments, the subject is human. In some embodiments, the administration of the composition is parenteral, oral, inhalation, transdermal, intraocular, iontophoretic, or transmucosal.

In some embodiments, the defective mucosal barrier disease or disorders is necrotizing enterocolitis (NEC). In some embodiments, the subject is a premature infant or newborn.

In some embodiments, the mucus barrier is in the gastrointestinal tract, the respiratory airway, the cervicovaginal surface, or ocular surface.

### BRIEF DESCRIPTION OF DRAWINGS

FIG. 1A is a graph showing the time-averaged mean squared displacement (MSD) of amine-modified microspheres in porcine intestinal mucus. The amine-modified microspheres were diluted in maleate buffer (MB), model bile (BS/PL), or mucus strengthening solution (FED).

FIG. 1B is a graph showing the time-averaged mean squared displacement (MSD) of carboxylate-modified microspheres in porcine intestinal mucus. The carboxylate-modified microspheres were diluted in maleate buffer (MB), model bile (BS/PL), or mucus strengthening solution (FED).

FIG. 1C is a graph showing the time-averaged mean squared displacement (MSD) of sulfate-modified microspheres in porcine intestinal mucus. The sulfate-modified microspheres were diluted in maleate buffer (MB), model bile (BS/PL), or mucus strengthening solution (FED).

FIG. 1D is a graph showing the ensemble-average (D_{eff}) of amine-modified microspheres in porcine intestinal mucus. The amine-modified microspheres were diluted in maleate buffer (MB), model bile (BS/PL), or mucus strengthening solution (FED).

FIG. 1E is a graph showing the ensemble-average (D_{eff}) of carboxylate-modified microspheres in porcine intestinal mucus. The carboxylate-modified microspheres were diluted in maleate buffer (MB), model bile (BS/PL), or mucus strengthening solution (FED).

FIG. 1F is a graph showing the ensemble-average (D_{eff}) of sulfate-modified microspheres in porcine intestinal mucus. The sulfate-modified microspheres were diluted in maleate buffer (MB), model bile (BS/PL), or mucus strengthening solution (FED).

FIG. 2 is a graph showing ensemble <MSD> versus time scale plots for carboxylate-modified microspheres in mouse intestinal explants.

FIG. 3A is a graph showing the time-averaged mean squared displacement (MSD) of amine-modified microspheres in porcine intestinal mucus, wherein the mucus was exposed to [Ca²⁺] at 5 mM, 10 mM, and 20 mM.

FIG. 3B is a graph showing the time-averaged mean squared displacement (MSD) of carboxylate-modified microspheres in porcine intestinal mucus, wherein the mucus was exposed to [Ca²⁺] at 5 mM, 10 mM, and 20 mM.

FIG. 3C is a graph showing the time-averaged mean squared displacement (MSD) of sulfate-modified microspheres in porcine intestinal mucus, wherein the mucus was exposed to [Ca²⁺] at 5 mM, 10 mM, and 20 mM.

FIG. 3D is a graph showing the ensemble-average (D_{eff}) of amine-modified microspheres in porcine intestinal mucus, wherein the mucus was exposed to [Ca²⁺] at 5 mM, 10 mM, and 20 mM.

FIG. 3E is a graph showing the ensemble-average (D_{eff}) of carboxylate-modified microspheres in porcine intestinal mucus, wherein the mucus was exposed to [Ca²⁺] at 5 mM, 10 mM, and 20 mM.

FIG. 3F is a graph showing the ensemble-average (D_{eff}) of sulfate-modified microspheres in porcine intestinal mucus, wherein the mucus was exposed to [Ca²⁺] at 5 mM, 10 mM, and 20 mM.

FIG. 4A is graph showing zeta potential of amine-, carboxylate-, and sulfate-microspheres in maleate buffer, model bile (BS/PL), and mucus barrier strengthening solution (FED).

FIG. 4B is graph showing zeta potential of amine-, carboxylate-, and sulfate-microspheres in maleate buffer with [Ca⁺⁺] concentrations at 5 mM, 10 mM, and 20 mM.

FIG. 4C is graph showing zeta potential of amine-, carboxylate-, and sulfate-microspheres in maleate buffer with pH at 3.5, 5.5, and 6.5.

FIG. 5A is graph showing particle size of amine-, carboxylate-, and sulfate-microspheres in maleate buffer, model bile (BS/PL), and mucus barrier strengthening solution (FED).

FIG. 5B is graph showing particle size of amine-, carboxylate-, and sulfate-microspheres in maleate buffer with [Ca++] concentrations at 5 mM, 10 mM, and 20 mM.

FIG. 5C is graph showing particle size of amine-, carboxylate-, and sulfate-microspheres in maleate buffer with pH at 3.5, 5.5, and 6.5.

FIG. 6A is a graph showing the time-averaged mean squared displacement (MSD) of amine-modified microspheres in porcine intestinal mucus at pH levels of 6.5, 5.5, and 3.5.

FIG. 6B is a graph showing the time-averaged mean squared displacement (MSD) of carboxylate-modified microspheres in porcine intestinal mucus at pH levels of 6.5, 5.5, and 3.5.

FIG. 6C is a graph showing the time-averaged mean squared displacement (MSD) of sulfate-modified microspheres in porcine intestinal mucus at pH levels of 6.5, 5.5, and 3.5.

FIG. 6D is a graph showing the ensemble-average (D_{eff}) of amine-modified microspheres in porcine intestinal mucus at pH levels of 6.5, 5.5, and 3.5.

FIG. 6E is a graph showing the ensemble-average (D_{eff}) of carboxylate-modified microspheres in porcine intestinal mucus at pH levels of 6.5, 5.5, and 3.5.

FIG. 6F is a graph showing the ensemble-average (D_{eff}) of sulfate-modified microspheres in porcine intestinal mucus at pH levels of 6.5, 5.5, and 3.5.

FIG. 7 is a graph showing the average speed of E. coli across mucus exposed to maleate buffer with [Ca⁺⁺] at 0 mM, 10 mM, and 20 mM or mucus barrier strengthening composition (FED).

### DETAILED DESCRIPTION

It is to be appreciated that certain aspects, modes, embodiments, variations and features of the invention are described below in various levels of detail in order to provide a substantial understanding of the present invention. The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural references unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, “biological agent” refers to a natural compound. In some embodiments, the biological agent is combined with a mucus strengthening composition. By way of example, but not by way of limitation, biological agents include, but are not limited to, e.g., mucin (e.g., MUC1), oligosaccharides (e.g., disialyllacto-N-tetraose (DSLNT)), trefoil factor 3 (TFF3), resistin-like molecule-β (RELM-β), RegIIIγ, anti-microbial peptides (AMPs), lysozyme, calcium chloride, lactoferrin, immunoglobulin A (IgA), immunoglobulin G (IgG), arachidonic (AA), docosahexaenoic acid (DHA), lactoferrin, apo-lactoferrin, and lysozymes. Biological agents can be isolated from a natural source, recombinantly produced or synthesized in vitro.

As used herein, “effective amount” or “therapeutically effective amount” refers to a quantity sufficient to achieve a desired therapeutic and/or prophylactic effect, e.g., an amount which eliminates, reduces, or ameliorates symptoms associated with diseases or disorders that exhibit or are a result of a defective mucosal barrier.

As used herein, “prevention” or “preventing” of a defective mucosal barrier and/or diseases or disorders that exhibit or are a result of a defective mucosal barrier refers to a composition that, in a statistical sample, reduces the occurrence of a defective mucosal barrier in the treated sample relative to an untreated control sample, or delays or prevents the onset of one or more symptoms of the disorder or condition relative to the untreated control sample. As used herein, preventing a defective mucosal barrier or a disease or disorder that exhibit or are a result of a defective mucosal barrier includes preventing the initiation of a defective mucosal barrier, delaying the initiation of a defective mucosal barrier, delaying the progression or advancement of a defective mucosal barrier or disease or disorder associated therewith. As used herein, prevention of a defective mucosal barrier or disease or disorder associated therewith, also includes preventing an occurrence or re-occurrence of a defective mucosal barrier or the disease or disorder.

As used herein, the term “strengthening the mucus barrier” refers to decreasing the permeability of a mucus barrier. In some embodiments, decrease in permeability is measured as a reduction in the speed of transport of a molecule, compound, or microbe across the mucus barrier. In some embodiments, decrease in permeability is measured as the prevention of transport of a molecule, compound, or microbe across the mucus barrier. In some embodiments, the pore size of the mucus barrier is decreased. Alternatively, or additionally, in some embodiments, strengthening the mucus barrier refers to increasing the depth of the mucus barrier. Alternatively, or additionally, in some embodiments, strengthening the mucus barrier refers to increasing the viscoelasticity of the mucus barrier.

As used herein, the terms “treating” or “treatment” or “alleviation” refers to therapeutic treatment, wherein the object is to slow down (lessen), reverse, or treat a defective mucosal barrier and/or diseases or disorders that exhibit or are a result of a defective mucosal barrier and their associated symptoms. A subject is successfully “treated” for a defective mucosal barrier or disease or disorder that exhibits or is a result of a defective mucosal barrier if, for example, after receiving a therapeutic amount of the mucus barrier strengthening composition according to the methods described herein, the subject shows observable and/or measurable reduction in or absence of one or more signs and/or symptoms of a diseases or disorders that exhibit or are a result of a defective mucosal barrier. It is also to be appreciated that the various modes of treatment or prevention of medical conditions as described are intended to mean “substantial,” which includes total but also less than total treatment or prevention, and wherein some biologically or medically relevant result is achieved.

### Mucus Barriers

General

Mucus is a complex gel network that forms a barrier between epithelial surfaces and the external environment. Mucus includes a highly branched protein network, the major structural component of which is mucin glycoproteins. Mucus also contains ˜2% (by mass) lipids and 90-95% by mass salts, cells, electrolytes, other cellular debris and water. Mucus functions as a robust barrier to control the passage of harmful molecules and organisms such as bacteria and function to facilitate efficient transport of nutrients across the epithelium.

Intestinal mucus barriers play a role in controlling the transport of intestinal lumen contents, including microbes, to underlying epithelium. Mucus control of microbial invasion is dependent on its structure and composition. Ultrastructural analysis of healthy intestinal mucus shows a heterogeneous nano- to micro-scale porous network, which acts as an effective barrier to intestinal microbes. A heterogeneous micro- to nano-scale network provides a semi-permeable barrier, in terms of size exclusion, to micro-scale microbial species. In certain disease states (e.g., Crohn's disease), altered intestinal mucus structure (e.g., pore size) together with compositional differences that may impact interactions of microbes with mucus may contribute to enhanced permeability.

Mucus barriers in intestines of premature infants do not prevent transport of microbes and/or microbial agents to the underlying epithelium to the same extent as mucus in full term infants, resulting in atypical infection with intestinal microbes, which can lead to necrotizing enterocolitis (NEC).

In general, the present technology relates to compositions and methods that are useful for strengthening the mucus barrier.

Mucus Barrier Strengthening Compositions

In some embodiments, the mucus barrier strengthening compositions include a model bile. In some embodiments, the mucus barrier strengthening compositions includes a model bile and a lipid mixture, wherein the lipid mixture is intermixed with the model bile.

In some embodiments, the model bile includes at least one salt and a lecithin. By way of example, but not by way of limitation, in some embodiments, the salt includes, but is not limited to sodium taurodeoxycholate (NaTDC), calcium-containing salts, calcium chloride (CaCl₂), acetate, carbonate, chloride, glubionate, gluceptate, gluconate, lactate, lactobionate, phosphate salts, NaCl, KCl, NaOH, Triz-maleate, bile salts, e.g., sodium glycocholate (NaGC), sodium glycochenodeoxycholate (NaGCDC), sodium glycodeoxycholate (NaGDC), sodium taurocholate (NaTC), sodium chenodeoxycholate (NaCDC), sodium taurodeoxycholate (NaTDC), and porcine bile extract.

By way of example, but not by way of limitation, in some embodiments, the lecithin includes, but is not limited to phosphoric acid, choline, fatty acids, glycerol, glycolipids, triglycerides, lysolecithin, and phospholipids. In some embodiments, the phospholipid includes, but is not limited to, e.g., phosphatidylcholine, phosphatidylethanolamine, phosphatidylinositol, and phosphatidic acid.

In some embodiments, the model bile also includes cholesterol and lysophosphadylcoline

In some embodiments, the lecithin is derived from animal or plant tissue. In some embodiments, the lecithin is extracted from soybeans, eggs, milk, marine sources, rapeseed, cottonseed, and sunflower. In some embodiments, the lecithin is hydrolyzed.

In some embodiments, the lipid mixture includes, but is not limited to, soybean oil, sodium oleate, 1-oleoyl-rac-glycerol, long chain triglycerides (e.g., monoolein and oleic acid), glycerol, glyceryl, olive oil, self-emulsifying drug delivery systems (SEDDS)(e.g., various formulations composed of vitamin E, Tween 80, labrasol, captex 355, ethyl alcohol in different proportions), self-emulsifying drug delivery systems (SNEDDS)(e.g., 30% w/w sesame oil, 30% w/w maisine 35-1, 30% w/w cremophor RH 40, 10% w/w ethanol), or a combination thereof.

In some embodiments, the model bile to lipid mixture ration is about 2:1, or about 3:1, or about 4:1, or about 5:1, or about 6:1.

In some embodiments, the mucus barrier strengthening composition also includes a pH lowering agent. In some embodiments, the pH lowering agent reduces in the pH in the mucus barrier to between about 3.0 to 5.5, or between about 3.5 to 5.0, or between about 4.0 to 4.5.

In some embodiments, the mucus barrier strengthening composition also includes one or more biological agents. By way of example, but not by way of limitation, in some embodiments, the biological agents enhance the strengthening of the mucus barrier. Biological agents include, but are not limited to, e.g., mucin (e.g., MUC1), oligosaccharides (e.g., disialyllacto-N-tetraose (DSLNT)), trefoil factor 3 (TFF3), resistin-like molecule-β (RELM-β), RegIIIγ, anti-microbial peptides (AMPs), lysozyme, calcium chloride, lactoferrin, immunoglobulin A (IgA), immunoglobulin G (IgG), arachidonic (AA), docosahexaenoic acid (DHA), lactoferrin, apo-lactoferrin, and lysozymes.

By way of example, but by way of limitation, Table 1 shows exemplary types and concentrations of biological agents that can be added to a mucus barrier strengthening composition.

TABLE 1
Additional Biological Agents

| Biological Agent | Concentration | |
|---|---|---|
| AA | 20-40 | mg/L |
| DHA | 10-30 | mg/L |
| apo-lactoferrin | 30-70 | g/L |
| IgA | 0.5-1.5 | g/L |
| IgG | 5-15 | mg/L |
| lysozyme | 2.5-7.5 | g/L |
| DSLNT | 2.5-7.5 | g/L |
| CaCl2 | 0.2-0.4 | g/L |
| mucin | 0.35-1.05 | mg/L |
| TFF3 | 0.13-0.4 | g/L |
| RELM-β | 0.13-0.4 | g/L |

In some embodiments, the composition also includes a maleate buffer. By way of example, but not by way of limitation, in some embodiments, the maleate buffer include Triz-ma, sodium chloride (NaCl), calcium chloride (CaCl₂), sodium azide (NaN₃), and sodium hydroxide (NaOH). In some embodiments, the maleate buffer has a pH about 3 to 6.5, or about 3.5 to 6.0, or about 4.0 to 5.5, or about 4.4 to 5.0. In some embodiments, includes about 0 to 50 mM, or about 5 to 45 mM, or about 10 to 40 mM, or about 15 to 35 mM, or about 20 to 30 mM, or about 23 to 28 mM of CaCl2. In some embodiments, includes about 0 to 50 mM, or about 5 to 45 mM, or about 10 to 40 mM, or about 15 to 35 mM, or about 20 to 30 mM, or about 23 to 28 mM of NaOH.

By way of example, but not by way of limitation, in some embodiments, the composition is mixture of a maleate buffer including about 100 mM Triz-ma, about 65 mM NaCl, between about 5-20 mM CaCl₂, about 3 mM NaN₃, and between about 0-40 mM NaOH, a model bile including about 12 mM NaTDC and about 4 mM lecithin, and a lipid mixture including about 35 mM soybean oil, about 30 mM soybean oleate, and about 15 mM 1-oleoyl-rac-glycerol.

Method of Making Mucus Strengthening Compositions

In some embodiments, a method for making the mucus strengthening compositions includes combining a salt and a lecithin to make a model bile. In some embodiments, the salt and the lecithin are dissolved in a solution. By way of example, but not by limitation, the solution includes, but is not limited to water, de-ionized water, PBS, and a buffer. In some embodiments the buffer is a maleate buffer.

By way of example, but not by way of limitation, in some embodiments, the maleate buffer includes Triz-ma, sodium chloride (NaCl), calcium chloride (CaCl₂), sodium azide (NaN₃), and sodium hydroxide (NaOH). In some embodiments, the maleate buffer has a pH about 3 to 6.5, or about 3.5 to 6.0, or about 4.0 to 5.5, or about 4.4 to 5.0. In some embodiments, includes about 0 to 50 mM, or about 5 to 45 mM, or about 10 to 40 mM, or about 15 to 35 mM, or about 20 to 30 mM, or about 23 to 28 mM of CaCl₂. In some embodiments, includes about 0 to 50 mM, or about 5 to 45 mM, or about 10 to 40 mM, or about 15 to 35 mM, or about 20 to 30 mM, or about 23 to 28 mM of NaOH.

By way of example, but not by way of limitation, in some embodiments, the salt includes, but is not limited to sodium taurodeoxycholate (NaTDC), calcium-containing salts, calcium chloride (CaCl2), acetate, carbonate, chloride, glubionate, gluceptate, gluconate, lactate, lactobionate, phosphate salts, NaCl, KCl, NaOH, Triz-maleate, bile salts, e.g., sodium glycocholate (NaGC), sodium glycochenodeoxycholate (NaGCDC), sodium glycodeoxycholate (NaGDC), sodium taurocholate (NaTC), sodium chenodeoxycholate (NaCDC), sodium taurodeoxycholate (NaTDC), and porcine bile extract.

By way of example, but not by way of limitation, in some embodiments, the lecithin includes, but is not limited to phosphoric acid, choline, fatty acids, glycerol, glycolipids, triglycerides, lysolecithin, and phospholipids. In some embodiments, the phospholipid includes, but is not limited to, e.g., phosphatidylcholine, phosphatidylethanolamine, phosphatidylinositol, and phosphatidic acid.

In some embodiments, the model bile also includes cholesterol and lysophosphadylcoline

In some embodiments, the lecithin is derived from animal or plant tissue. In some embodiments, the lecithin is extracted from soybeans, eggs, milk, marine sources, rapeseed, cottonseed, and sunflower. In some embodiments, the lecithin is hydrolyzed.

In some embodiments, a method for making mucus strengthening compositions also includes adding a lipid mixture to the model bile. In some embodiments, the model bile to lipid mixture ration is about 2:1, or about 3:1, or about 4:1, or about 5:1, or about 6:1.

In some embodiments, the lipid mixture includes, but is not limited to, soybean oil, sodium oleate, 1-oleoyl-rac-glycerol, long chain triglycerides (e.g., monoolein and oleic acid), glycerol, glyceryl, olive oil, self-emulsifying drug delivery systems (SEDDS)(e.g., various formulations composed of vitamin E, Tween 80, labrasol, captex 355, ethyl alcohol in different proportions), self-emulsifying drug delivery systems (SNEDDS)(e.g., 30% w/w sesame oil, 30% w/w maisine 35-1, 30% w/w cremophor RH 40, 10% w/w ethanol), or a combination thereof or a combination thereof.

In some embodiments, the mucus barrier strengthening composition also includes a pH lowering agent. In some embodiments, the pH lowering agent reduces in the pH in the mucus barrier to between about 3.0 to 5.5, or between about 3.5 to 5.0, or between about 4.0 to 4.5.

In some embodiments, the mucus barrier strengthening composition also includes one or more biological agents. By way of example, but not by way of limitation, in some embodiments, the biological agents enhance the strengthening of the mucus barrier. Biological agents include, but are not limited to, e.g., mucin (e.g., MUC1), oligosaccharides (e.g., disialyllacto-N-tetraose (DSLNT)), trefoil factor 3 (TFF3), resistin-like molecule-β (RELM-β), RegIIIγ, anti-microbial peptides (AMPs), lysozyme, calcium chloride, lactoferrin, immunoglobulin A (IgA), immunoglobulin G (IgG), arachidonic (AA), docosahexaenoic acid (DHA), lactoferrin, apo-lactoferrin, and lysozymes.

### Methods of Use for Mucus Barrier Strengthening Compositions

In some embodiments, the mucus barrier strengthening compositions disclosed herein are administered to decrease or prevent the transport of molecules, compounds, or microbes across a mucus barrier. In some embodiments, the compound is a drug or a drug carrier. In some embodiments, the microbe is a bacteria.

In some embodiments, the mucus barrier strengthening compositions are administered to increase the viscoelasticity of a mucus membrane.

In some embodiments, the mucus barrier strengthening compositions are administered to alter the mucosal structure of a mucus barrier. In some embodiments, the mucus barrier strengthening compositions reduces the pore size of the mucosal structure. Additionally, or alternatively, in some embodiments, the mucus barrier strengthening compositions reduces the permeability of the mucus barrier.

In some embodiments, the mucus barrier strengthening compositions are administered to increases the depth of a mucus barrier. In some embodiments, the mucus barrier depth is increased to between about 10 to 200 μm, or about 20 to 180 μm, or about 30 to 160 μm, or about 40 to 140 μm, or about 50 to 120 μm, or about 60 to 100 μm, or about 70 to 80 μm.

In some embodiments, the mucosal barrier is in the gastrointestinal tract, the respiratory airway, the cervicovaginal surface, or ocular surface.

### Prophylactic and Therapeutic Uses of Mucus Barrier Strengthening Compositions

General

In some embodiments, the mucus barrier strengthening compositions described herein are useful to prevent or treat a defective mucosal barrier and/or diseases or disorders that exhibit or are a result of a defective mucosal barrier. Diseases or disorders that exhibit or are a result of a defective mucosal barrier, include but are not limited to necrotizing enterocolitis (NEC), Crohn's disease, oral cancer, inflammatory bowel disease, mucositis, vesiculo-erosive conditions, infections, neuropathic pain and salivary dysfunction. In some embodiments, the defective mucosal barrier disease or disorder is NEC. In some embodiments, a subject with NEC has an intestinal mucosal barrier that is reduced, missing, or easily traversed, e.g., by bacteria or other pathogens (e.g., permissive to transport of substances, e.g., microbes, to the underlying epithelium). Accordingly, the present methods provide for the prevention and/or treatment of a defective mucosal barrier and/or diseases or disorders that exhibit or are a result of a defective mucosal barrier in a subject by administering an effective amount of a mucus barrier strengthening composition to a subject in need thereof. In some embodiments, the subject is a mammal. In some embodiments, the subject is human. In some embodiments, the subject is a human newborn, infant, or premature infant.

Therapeutic Methods

In some embodiments, the present technology includes methods for treating diseases or disorders that exhibit or are a result of a defective mucosal barrier for therapeutic purposes. In therapeutic applications, a mucus barrier strengthening composition is administered to a subject suspected of having or already suffering from a disease or disorder that exhibits or is a result of a defective mucosal barrier in an amount sufficient to cure, or at least partially arrest, the symptoms of the disease, including its complications and intermediate pathological phenotypes in development of the disease. As such, in some embodiments, the present technology provides methods of treating an individual having or suspected of having diseases or disorders that exhibit or are a result of a defective mucosal barrier.

Subjects suffering from diseases or disorders that exhibit or are a result of a defective mucosal barrier can be identified by any or a combination of diagnostic or prognostic assays known in the art. By way of example, but not by way of limitation, typical symptoms of NEC include, but are not limited to, abdominal distention and tenderness, pneumatosis intestinalis, blood in stools, intestinal gangrene, bowel perforation, sepsis, shock, or a combination thereof.

Prophylactic Methods

In some aspects, the technology disclosed herein provides a method for preventing or reducing the likelihood or severity of a defective mucosal barrier and/or diseases or disorders that exhibit or are a result of a defective mucosal barrier in a subject having or suspected of having a disease or disorder that exhibits or is a result of a defective mucosal barrier. Subjects at risk for diseases or disorders that exhibit or are a result of a defective mucosal barrier can be identified by any or a combination of diagnostic or prognostic assays known in the art. By way of example, but not by way of limitation, subjects at risk for NEC include, but are not limited to, premature infants. By way of example, but not by way of limitation, subjects at risk for diseases or disorders that result from a defective mucosal barrier include, but are not limited to, people between the ages 15-30 that have symptoms on Crohn's Disease and inflammatory bowel diseases and people who smoke. In prophylactic applications, a mucus barrier strengthening composition is administered to a subject susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In some embodiments, the subject is a newborn or premature infant.

### Modes of Administration and Effective Dosages

Any method known to those in the art for contacting a mucosal barrier with a composition may be employed. Suitable methods include in vitro, ex vivo, or in vivo methods. In vivo methods typically include the administration of a mucus barrier strengthening composition, such as those described above, to a mammal, suitably a human. When used in vivo for therapy, the mucus barrier strengthening composition is administered to the subject in effective amounts (i.e., amounts that have desired therapeutic effect). The dose and dosage regimen will depend upon the disease stage of the subject, the subject, and the subject's history.

The effective amount may be determined during pre-clinical trials and clinical trials by methods familiar to physicians and clinicians. An effective amount of a mucus barrier strengthening composition useful in the methods may be administered to a mammal in need thereof by any of a number of well-known methods for administering pharmaceutical compounds. The composition may be administered systemically or locally.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. In all cases, a composition for parenteral administration must be sterile and should be fluid to the extent that easy syringability exists. It should be stable under the conditions of manufacture and storage and must be preserved against the contaminating action of microorganisms such as bacteria and fungi.

Oral compositions generally include an inert diluent or an edible carrier. For the purpose of oral therapeutic administration, the active compound can be incorporated with excipients and used in the form of liquids, tablets, troches, or capsules, e.g., gelatin capsules.

For administration by inhalation, the compounds can be delivered in the form of an aerosol spray from a pressurized container or dispenser, which contains a suitable propellant, e.g., a gas such as carbon dioxide, or a nebulizer.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### EXAMPLES

The following examples are provided to more fully illustrate various implementations of the present technology. These examples should in no way be construed as limiting the scope of the present technology.

### Example 1

### Mucus Barrier Strengthening Composition Reduces Particle Transport Through Gastrointestinal Mucus

This examples shows that the mucus barrier strengthening composition reduces particle transport through gastrointestinal mucus.

Methods and Material

Preparation and Characterization of Microspheres and Mucus Barrier Strengthening Solution:

The following is an exemplary method for preparing a mucus barrier strengthening composition. The following exemplary method is a method for preparing the mucus barrier strengthening composition as a solution (hereinafter referred to as “mucus barrier strengthening solution”). The concentrations for the exemplary embodiment of the various solutions, i.e., maleate buffer, model bile, and mucus barrier strengthening solution, are found in Table 2.

1 L of maleate buffer (MB) solution was prepared by adding 23.7 g of Triz-Ma (final concentration 100 mM), 3.8 g of NaCl (final concentration 65 mM), 1.1 g of CaCl₂ (final concentration 10 mM), and 0.195 g of NaN3 (final concentration 3 mM) into a 1 L volumetric flask. De-ionized water was added until the solution for a total volume of 1 L at room temperature. The pH of the maleate buffer was adjusted to 6.5 by adding 1.6 g of NaOH (final concentration 40 mM). The solution was stirred at room temperature.

In some embodiments, the 1 L maleate buffer was prepared with different CaCl₂ concentrations to vary the amount of calcium ion, e.g., at 5 mM (0.55 g of CaCl₂) and 20 mM (2.2 g of CaCl₂).

In some embodiments, the 1 L of maleate buffer solution was prepared with different pH's, e.g., at pH 3.5 by not adding NaOH, or prepared at pH 5.5 by adding 1.45 g of NaOH (final concentration 25 mM).

Model bile (BS/PL) was prepared adding 0.313 g of NaTDC (final concentration 12 mM) and 0.15 g Lecithin (final concentration 4 mM) to 50 ml of maleate buffer solution. The complete model bile solution was stirred at 300 RPM for 12 hours at room temperature. The model bile had a pH of 6.5.

The mucus barrier strengthening solution (FED) was prepare by adding 1.6 mL of soybean oil (final concentration 35 mM), 0.46 g of sodium oleate (final concentration 30 mM), and 0.27 g of 1-Oleoyl-rac-glycerol (final concentration 15 mM) to 50 ml of the model bile. The complete mucus barrier strengthening solution was stirred at 300 RPM at room temperature. The mucus barrier strengthening solution had a pH of 6.5.

Fluorescently labeled yellow-green FluoSpheres (Invitrogen Molecular Probes, Carlsbad, Calif.) were used to prepare modified microspheres Amine-, carboxylate-, and sulfate-modified microspheres (2% solids in distilled water with 2 mM azide), 200 nm in diameter, were diluted in a mucus barrier strengthening solution (FED), maleate buffer (MB), and model bile (BS/PL) for a final particle concentration of 0.0025 wt.-%.

Serial dilutions at room temperature of each modified microsphere were performed as follow:

Dilution 1: 975 μl of solution (i.e., MB, BS/PL, or FED)+25 μl modified microsphere (i.e., amine-, carboxylate-, and sulfate-).

Dilution 2: 950 μl of solution (i.e., MB, BS/PL, or FED)+50μ of Dilution 1.

In some embodiments, modified microsphere diluted in maleate buffer with CaCl₂ at 10 mM and NaOH at 40 mM concentrations were used as a control parameter. In some embodiments, modified microsphere diluted in maleate buffer with varying CaCl₂ concentrations, e.g., 5 mM, 10 mM, and 20 mM in maleate buffer at pH 6.5 were used to observe calcium ion effect on particle diffusion in mucus. In some embodiments, modified microsphere diluted in maleate buffer with different pHs, e.g., at a pH of 3.5, 5.5, or 6.5 were used to observe the effect of pH influence on particle diffusion.

TABLE 2
Exemplary Mucus Barrier Strengthening Solution

| | | | |
|---|---|---|---|
| Maleate Buffer | Triz-ma | 100 | mM |
| | NaCl | 65 | mM |
| | CaCl₂ | 5-20 | mM |
| | NaN₃ | 3 | mM |
| | NaOH | 0-40 | mM |
| Model Bile | NaTDC | 12 | mM |
| | Lecithin | 4 | mM |
| Lipid Mixture | Soybean Oil | 35 | mM |
| | Sodium Oleate | 30 | mM |
| | 1-Oleoyl-rac-glycerol | 15 | mM |

The modified microsphere sizes and zeta potentials (ζ-potentials) of the modified microsphere were determined using dynamic light scattering.

Native Mucus Collection and Preparation:

Porcine intestines (Research 87, Boylston, Mass.) were obtained from a local abattoir within 2 hours of slaughter. Native mucus was scraped with a spatula from pig jejunum and stored at −80° C. until use.

Ex Vivo Preparation of Excised Mouse Intestine:

3 week old FVB/N type mice were euthanized via CO₂. Intestinal tissue segments, about 1 cm in length, were obtained from the intestines. The intestinal tissue segments were excised from about 10 cm from the exit of the stomach, which corresponds to the jejunum. Intestine fragments were cut open to expose the intestinal lumen, and placed into a chamber on a microscope slide maintained in a humidified environment.

Real-Time Multiple Particle Tracking (MPT) of Microspheres in Intestinal Mucus:

The trajectories of fluorescently labeled microspheres were captured using a 12.5 megapixel cooled Olympus DP70 digital color camera (Olympus, Center Valley, Pa.) mounted on an inverted Olympus IX51 microscope attached with X-Cite 120 fluorescence system (EXFO, Mississauga, Ontario, Canada).

10 μl of mucus barrier strengthening solution, maleate buffer, or model bile with modified microspheres (i.e., amine-, carboxylate-, and sulfate-modified microspheres) was deposited onto approximately 10 mm excised mouse explant tissue segments. The treated excised tissues were placed in a dark, humid chamber for 90 minutes at room temperature before imaging. Excised tissue obtained after treatments was placed within chambers formed by 0.8 mm deep silicone gaskets (Grace Bio-Labs) attached to microscope slides.

Scraped mucus was placed within non-fluorescent 8-well polystyrene medium chambers (Thermo Fisher Scientific, Rochester, N.Y.). 10 μl of mucus barrier strengthening solution, maleate buffer, or model bile with modified microspheres (i.e., amine-, carboxylate-, and sulfate-modified microspheres) was deposited onto approximately 200 μl of scraped native mucus. The treated scraped native mucus were placed in a dark, humid chamber for two hours at room temperature before imaging.

The mucosal specimens (i.e., scraped porcine mucus and explant tissue segments) post treatment were covered and equilibrated for 2 hours at 25° C. in a humid chamber prior to microscopy. Modified microsphere trajectories were recorded using Olympus DP imaging software with a frame rate of 30 fps for 20 seconds. Trajectories of n-100 modified microsphere were analyzed for each experiment and three experiments were performed from three different mucus specimens for each experimental setup to account for mucus variability. Trajectories for each particle type were generated using the feature point detection and tracking algorithm of the ParticleTracker ImageJ plugin developed by Sbalzarini et al., J. Struct. Biol., 151: 182-195 (2005). Particle coordinates were transformed into time-averaged mean squared displacements (MSD) as MSD=[x(t+τ)−x(t)]²+[y(t+τ)−y(t)]² and effective diffusivities (D_{eff}) as D_{eff}=MSD/(4τ) where x(t) and y(t) represent the nanoparticle coordinates at a given time, and τ is the time scale.

The extent of particle interaction with the mucus gel network was determined by fitting particle MSD vs. time scale to MSD=4D₀τ^{α}, where α is the anomalous exponent indicating particle motion obstruction and D₀ is time independent diffusion coefficient.

Analysis of Mucus Structure:

Lectin from Ulex europaeus agglutinin (UEA-1) conjugated with TRITC (Sigma Aldrich-L4889) stored in −20° C. were allowed to warm for about 15 minutes at room temperature. 10 μg/mL lectin working solution was prepared using the following serial dilutions: Dilution 1) 0.5 mg Lectin in 2 mL PBS and Dilution 2) 80 μl of Dilution 1 and 1920 μl PBS.

4 μl of 10 μg/ml lectin was added on top of 80 μl of scraped porcine mucus that were placed into 1.6 mm thick concavity slide. After the treated scraped porcine mucus were incubated for 20 minutes in a dark humid chamber at room temperature, 4 μl of yellow-green fluorescently labeled carboxylate-modified microspheres mixed in mucus barrier strengthening solution (described above) was added to the mucus and allowed to diffuse into the mucus for 2 hours in a dark humid chamber. Structure indicated via lectin staining and relative positions of mucus and carboxylate-modified microspheres were assessed using a Zeiss LSM 700 confocal microscope. Macro-scale changes in mucus structure were visually observed and imaged for each type of preparation.

Results

### Mucus Barrier Strengthening Solution Reduces Particle Transport Across Mucus Membranes

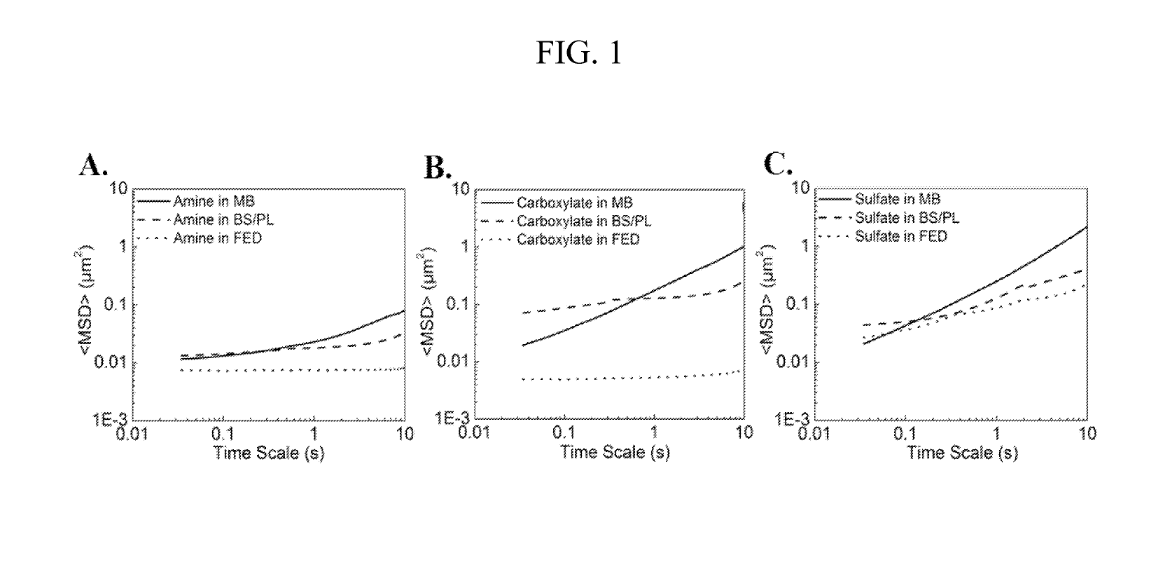

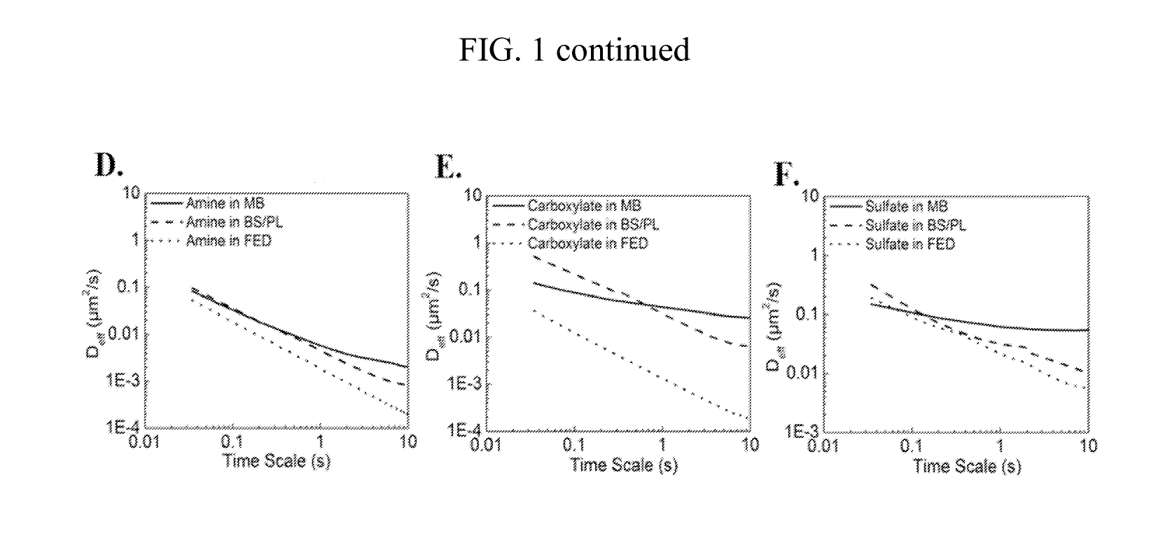

Mucus barrier strengthening solution hindered particle transport through collected porcine intestinal mucus as compared to modified microspheres in maleate buffer and model bile (FIGS. 1A-F). Coordinates of fluorescently labeled polystyrene microspheres with differing surface chemistry were captured using real-time MPT and transformed into time-averaged MSDs at various time scales (FIGS. 1A-C). The ensemble-average particle transport rates (D_{eff}) decreased with time, as expected in a heterogeneous medium such as mucus (FIG. 1D-F). At a time scale of 1 second, amine-, carboxylate- and sulfate modified microspheres in the mucus barrier strengthening solution were 3-, 30- and 2-fold slower, respectively, than modified microspheres in maleate buffer with [Ca²⁺]=10 mM (FIG. 1A-C). Average α values, reflective of diffusive (α=1) or sub-diffusive (α<1) motion, were reduced from 0.46 to 0.018, 0.74 to 0.09, and 0.87 to 0.36 for amine-, carboxylate-, and sulfate-modified microspheres, respectively, in the mucus barrier strengthening solution (FED) compared to maleate buffer (MB).

At a time scale of 10 seconds, the ensemble-average D_{eff} of amine-, carboxylate- and sulfate-modified microspheres were reduced 2.5, 4, and 5.5-fold, respectively, in the presence of model bile (BS/PL) (FIGS. 1D-F). However, the comparison of the effects of the mucus barrier strengthening solution to model bile shows the significant impact of the lipid mixture on reducing particle transport across the mucus barrier. Ensemble averaged effective diffusivities of modified microspheres at τ=10 seconds were hindered 4, 35, and 2-fold, respectively, in the mucus barrier strengthening solution relative to model bile.

Structural changes after treatment of porcine mucus were observed by the use of time scale-dependent displacements to quantify the frequency-dependent viscous or loss modulus, G″, and elastic or storage modulus G′. For all modified microspheres studied, gastrointestinal mucus poses higher elastic modulus than viscous modulus, indicating a gel-like network structure. Furthermore, the increase in elastic modulus with addition of a mucus barrier strengthening solution was greater than that of viscous modulus, indicating strengthening of the mucus barrier (data not shown).

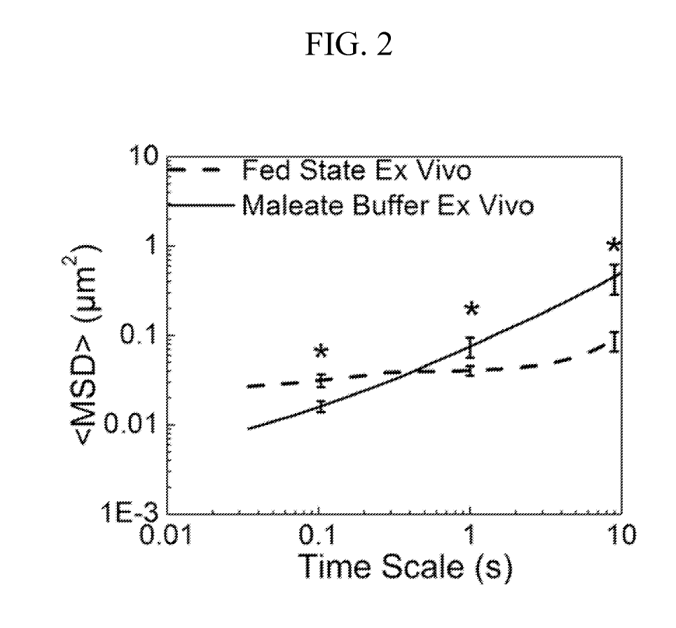

To determine if mucus barrier strengthening solution would have a similar effect on mucus that was intact on intestinal tissue, the mucosal surface of a mouse intestinal explant collected immediately after sacrifice was exposed to modified microspheres diluted in mucus barrier strengthening solution or maleate buffer. Intestinal contents characteristic of the mucus barrier strengthening solution reduced transport rates (as reflected in <MSD>) of microspheres over 5-fold compared to buffer at a time scale of 10 seconds (FIG. 2).

### Influence of [Ca²⁺] and pH on Particle Transport Across a Mucus Barrier

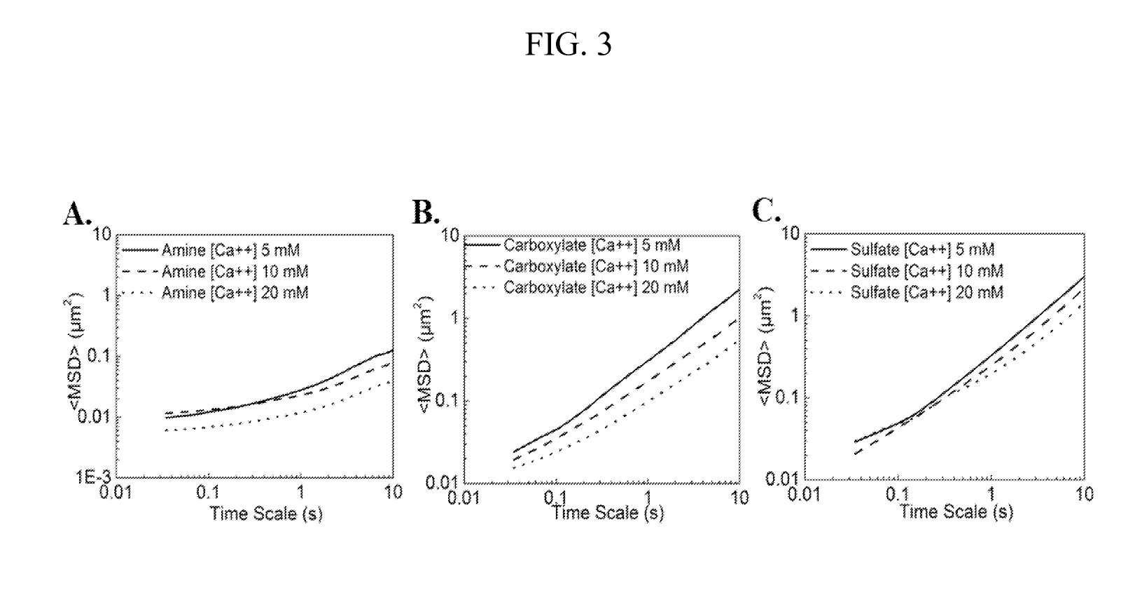

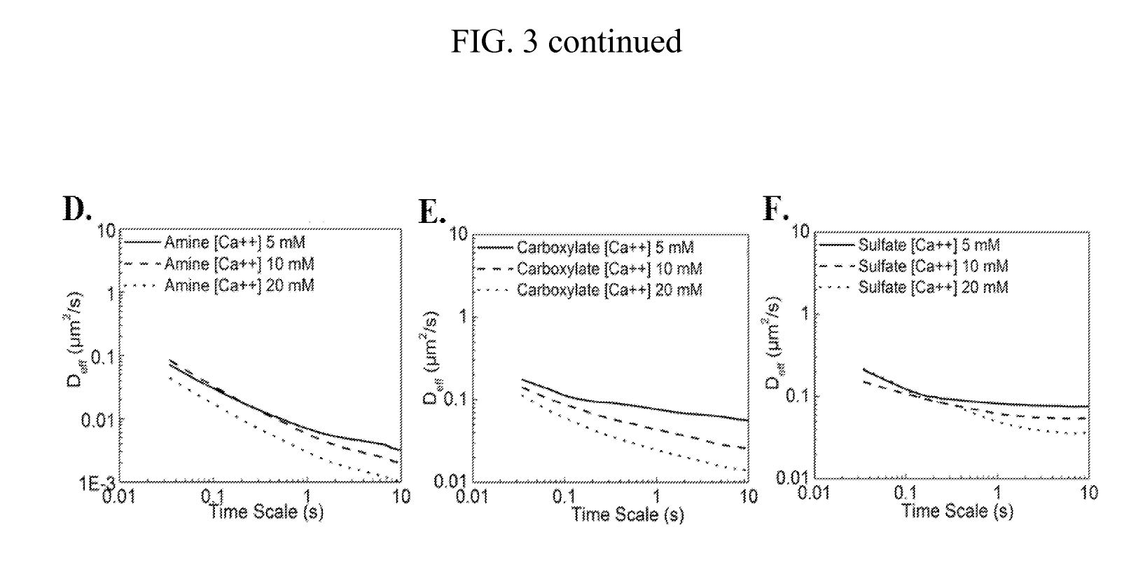

Increases in [Ca²⁺] and decreases in pH enhanced the mucus barrier strengthening solution's prevention of particle transport across mucus barriers. With increasing [Ca²⁺], the mobility of all types of modified microspheres in intestinal mucus decreased (FIGS. 3A-C).

At a time scale of 10 seconds, the ensemble-average effective diffusivity D_{eff} of amine-, carboxylate-, and sulfate-modified microspheres dosed at [Ca²⁺] of 20 mM were 3-, 4-, and 2-fold lower, respectively, than those of the same modified microspheres dosed at [Ca²⁺] of 5 mM (FIGS. 3D-F) in porcine intestinal mucus.

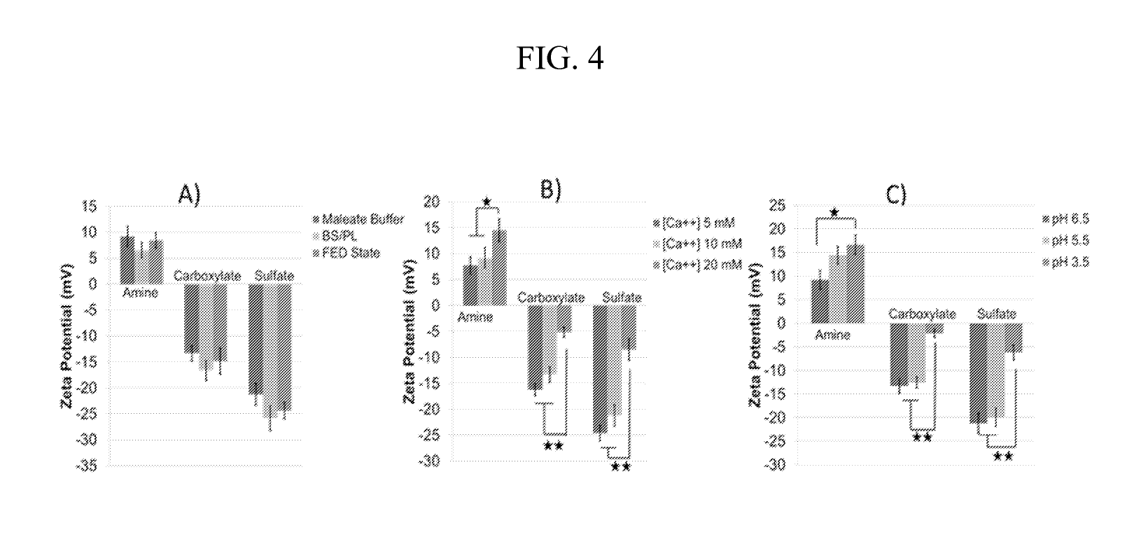

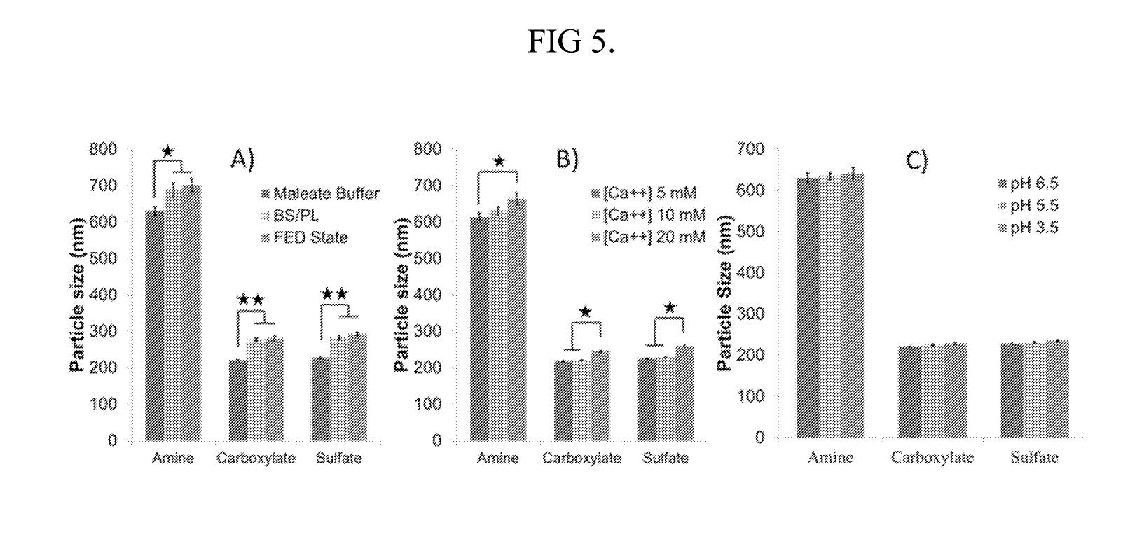

Given the similarity in reported mucus mesh and particle sizes, it is likely that enhanced mucin cross-linking and associated mucus structural changes are responsible in large part for observed impact of [Ca²⁺] on transport. However, impact of medium [Ca²⁺] on particle surface potential and size may also contribute. With an increase in [Ca²⁺] from 5 mM to 20 mM, zeta potential of cationic amine-modified microspheres increased from 7.74±1.65 mV to 14.56±2.19 and anionic carboxylate- and sulfate-modified particle zeta potentials decreased from −16.2±1.12 mV to −5.13±0.96 mV and −24.6±1.5 mV to −8.45±2.05 mV, respectively (FIG. 4B). Thus, electrostatic interactions between mucus proteins and modified microspheres were potentially responsible in part for decreases in amine-modified particle effective diffusivities from 0.0053 μm²/s to 0.0021 μm²/s and carboxylate- and sulfate-modified particle effective diffusivities from 0.065 μm²/s to 0.018 μm²/s and 0.079 μm²/s to 0.041 μm²/s, respectively (FIGS. 4A-C). Furthermore, effective diameters of all modified microspheres increased upon addition of [Ca²⁺] from 5 mM to 20 mM (FIGS. 5A-C).

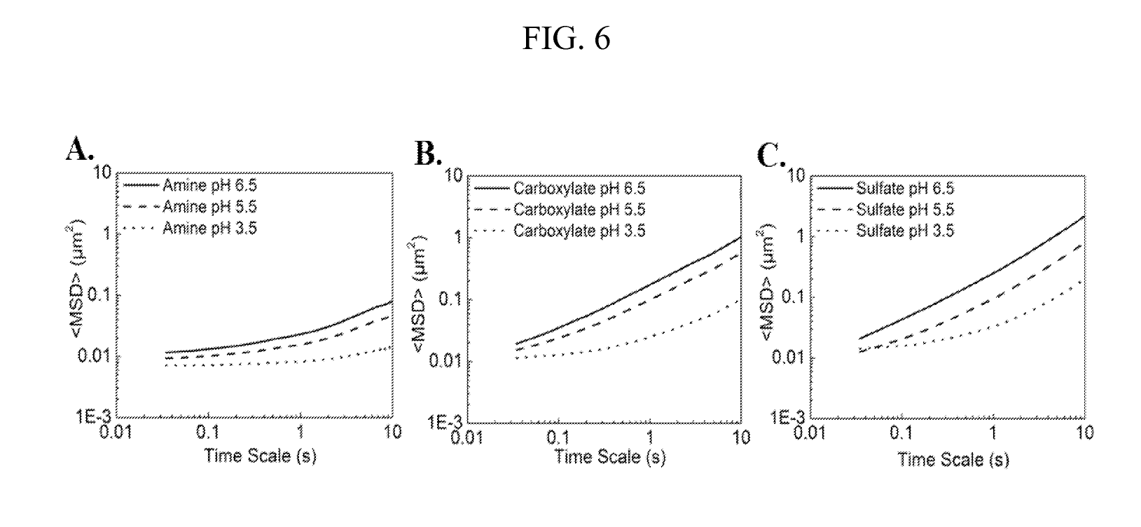

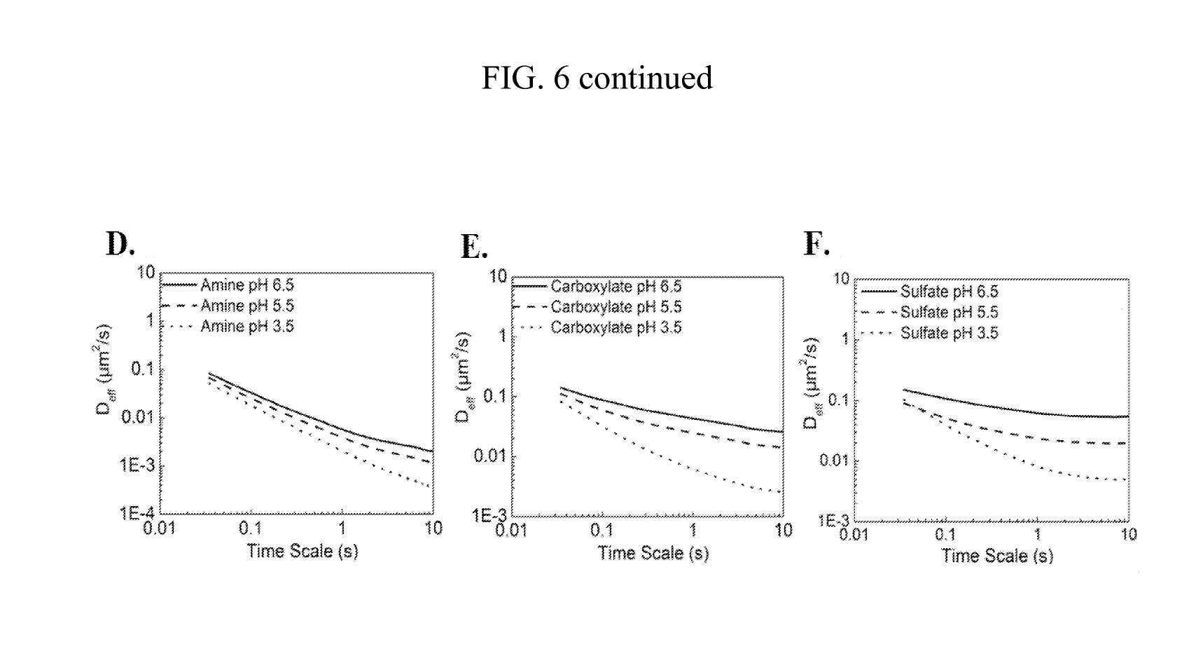

As the pH of the dosing solution decreases, particle mobility becomes more hindered (FIG. 6A-C). At a time scale of 10 seconds, amine-, carboxylate-, and sulfate-modified particle ensemble averaged effective diffusivities were reduced 3-, 6.5-, and 7-fold, respectively, with a decrease of pH from 6.5 to 3.5 (FIG. 6D-F).

Zeta potential measurements were moderately dependent upon the pH of the medium (FIG. 4C). Highly acidic environment (pH 3.5) significantly alters particle surface charge relative to pH 5.5 and 6.5, which could influence modified microsphere diffusivities. At pH 3.5, cationic amine-modified microspheres has the highest zeta potential (16.65±2.02 mV) and lowest ensemble-average Deff (0.0013 μm2/s). Similarly, anionic carboxylate- and sulfate-modified microspheres have surface charge closer to neutral (−2.12±0.8 mV and −6.15±1.6 mV) and the lowest effective diffusivities (0.0044 μm2/s and 0.0067 μm2/s), as expected due to electrostatic interactions between negatively charged mucus glycoproteins and modified microspheres.

These results show that the mucus barrier strengthening compositions of the present technology will reduce the transport of molecules across a mucus barrier. As such the mucus barrier strengthening compositions disclosed herein are useful for enhancing mucosal barriers.

### Example 2

### Mucus Barrier Strengthening Composition Reduces Microbe Transport to Underlying Epithelium

This example shows that treatment of a mucus barrier strengthening composition reduces microbe transport to underlying epithelium.

Methods and Material

Maleate buffer solutions at 0 mM, 10 mM and 20 mM concentration of CaCl₂ and mucus barrier strengthening solution (FED) as described in Example 1 were used in the following experiment.

E. coli (MG1655, ATCC 700926) was streaked onto agar plates three times to obtain single colonies to be inoculated into 10 ml Luria-Bertani (LB) medium. Single colonies were grown at 37° C. for 12 hours with vigorous shaking (300 rpm). Cell density was measured around 3×10⁹. E. coli was dosed into treatment solutions, i.e., maleate buffer solutions (10 mM or 20 mM CaCl₂) and mucus barrier strengthening solution, at 25 n1 treatment solution to 5 n1 E. coli in LB at about pH 6.5. Prepared microbe solutions were added onto scraped porcine native mucus with a volume ratio of 1/10 v:v at room temperature. Samples were kept in humid chamber for about 30 minutes before microscopy.

Diffusion of E. coli was monitored using an Olympus DP70 digital color camera (Olympus, Center Valley, Pa.) mounted on an inverted Olympus IX51 microscope with attached X-Cite 120 fluorescence illumination system (EXFO, Mississauga, Ontario, Canada). Bacteria diffusion videos were captured at 40× magnification with 512×512 pixel resolution (with 2×2 binning) with a frame rate of approximately 30 fps for 20 seconds. 220×200 pixel region of interest (ROI) selection was used to reduce the field of view and facilitate bacteria trajectory analysis. Individual bacteria trajectories and velocities were obtained using a modified version of the IDL colloid-tracking from Crocker et al., Journal of Colloid and Interface Science, 179: 298-310 (1996). The algorithm was edited to account for elongated modified microspheres instead of spherical ones since the E. coli are rod-shaped.

Results

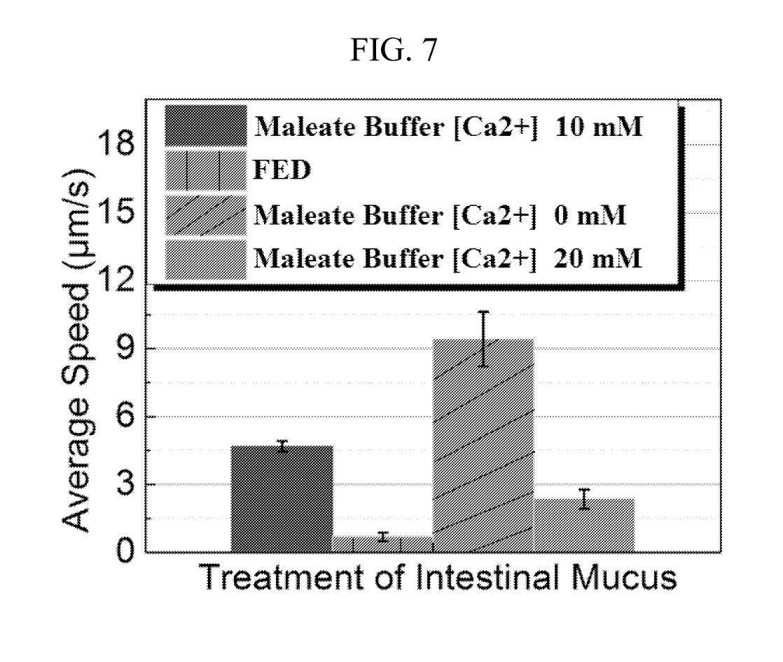

E. coli velocity in mucus treated with mucus barrier strengthening solution (FED) was reduced 8 fold as compared to maleate buffer with 0 mM [Ca²⁺] and about 2.5 fold as compared to maleate buffer with 20 mM [Ca²⁺] (FIG. 7).

These results show that the mucus barrier strengthening solution was effective a reducing the translocation of bacteria across a mucus barrier. Accordingly, the results show that mucus barrier strengthening compositions of the present technology are useful in reducing bacteria movement across a mucus barrier to the underlying epithelium. The reduced movement of bacteria could lead to the reducing of infection.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into sub-ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A composition comprising a model bile and a lipid mixture, wherein the model bile comprises at least one salt and a lecithin.

2. The composition of claim 1, wherein the at least one salt is selected from the group consisting of: bile salts, calcium-containing salts, sodium taurodeoxycholate (NaTDC), calcium chloride (CaCl2), acetate, carbonate, chloride, glubionate, gluceptate, gluconate, lactate, lactobionate, phosphate salts, NaCl, KCl, NaOH, Triz-maleate, bile salts, e.g., sodium glycocholate (NaGC), sodium glycochenodeoxycholate (NaGCDC), sodium glycodeoxycholate (NaGDC), sodium taurocholate (NaTC), sodium chenodeoxycholate (NaCDC), sodium taurodeoxycholate (NaTDC), and porcine bile extract.

3. The composition of claim 1, wherein the lecithin comprises one or more compounds selected from the group consisting of: phosphoric acid, choline, fatty acids, glycerol, glycolipids, triglycerides, lysolecithin, and phospholipids.

4. The composition of claim 1, wherein the lipid mixture comprises one or more of soybean oil, sodium oleate, and 1-oleoyl-rac-glycerol, and long chain triglycerides.

5. The composition of claim 1, further comprising a pH lowering agent.

6. The composition of claim 1, further comprising one or more biological agents, wherein the biological agent is selected from the group consisting of mucin, oligosaccharides, trefoil factor 3 (TFF3), resistin-like molecule-β (RELM-β), RegIIIγ, anti-microbial peptides (AMPs), lysozyme, lactoferrin, immunoglobulin A (IgA), immunoglobulin G (IgG), arachidonic (AA), docosahexaenoic acid (DHA), lactoferrin, and apo-lactoferrin.

7. The composition of claim 1, further comprising a maleate buffer.

8. A method for decreasing the permeability of a mucus barrier, the method comprising administering an effective amount of the composition of claim 1 to a subject.

9. The method of claim 8, wherein the mucus barrier is in the gastrointestinal tract, the respiratory airway, the cervicovaginal surface, or ocular surface.

10. The method of claim 8, wherein administration of the composition is parenteral, oral, inhalation, transdermal, intraocular, iontophoretic, or transmucosal.

11. The method of claim 8, wherein the subject is human.

12. A method for prevent or treating a defective mucosal barrier disease or disorders, the method comprising administering an effective amount of the composition of claim 1 to a subject in need thereof.

13. The method of claim 12, wherein the subject is human.

14. The method of claim 12, wherein administration of the composition is parenteral, oral, inhalation, transdermal, intraocular, iontophoretic, or transmucosal.

15. The method of claim 12, wherein the defective mucosal barrier disease or disorders is necrotizing enterocolitis (NEC).

16. The method of claim 15, wherein the subject is a premature infant or newborn.

17. The method of claim 12, wherein the mucus barrier is in the gastrointestinal tract, the respiratory airway, the cervicovaginal surface, or ocular surface.
